SEQUENCE LISTING (17 sequences)

SEQ ID NO: 1 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLLLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISCCIDPLIYYYASSECQRYVYSILCCKESSDPSSYNSSGQLMASKMDTCSSNLNNSIYKKLLT

SEQ ID NO: 2 - protein, 10 aa, Homo sapiens
NPNDKYEPFW

SEQ ID NO: 3 - protein, 20 aa, Homo sapiens
NPNDKYEPFWEDEEKNESGL

SEQ ID NO: 4 - protein, 30 aa, Homo sapiens
NPNDKYEPFWEDEEKNESGLTEYRLVSINK

SEQ ID NO: 5 - protein, 20 aa, Homo sapiens
NPNDKYEPFWEDEEKNESGL

SEQ ID NO: 6 - protein, 10 aa, Homo sapiens
TFLLRNPNDK

SEQ ID NO: 7 - protein, 480 aa, Homo sapiens [sequence omitted]

SEQ ID NO: 8 - protein, 6 aa, Homo sapiens
NPNDKY

SEQ ID NO: 9 - protein, 13 aa, Homo sapiens
NPNDKYEPFWEDE

SEQ ID NO: 10 - protein, 16 aa, Homo sapiens
NPNDKYEPFWEDEEKN

SEQ ID NO: 11 - protein, 4 aa, Homo sapiens
NPND

SEQ ID NO: 12 - protein, 5 aa, Homo sapiens
NPNDK

SEQ ID NO: 13 - protein, 6 aa, Homo sapiens
NPNDKY

SEQ ID NO: 14 - protein, 7 aa, Homo sapiens
NPNDKYE

SEQ ID NO: 15 - protein, 8 aa, Homo sapiens
NPNDKYEP

SEQ ID NO: 16 - protein, 9 aa, Homo sapiens
NPNDKYEPF

SEQ ID NO: 17 - protein, 12 aa, Homo sapiens
NPNDKYEPFWED

(19) United States
(12) Patent Application Publication
(10) Pub. No.: US 2021/0268062 A1
(43) Pub. Date: Sep. 2, 2021

(54) INFLAMMATORY SKIN DISORDER TREATMENT

(71) Applicant: NOVAPEP PTY LTD, New South Wales (AU)

(72) Inventors: Christopher John JACKSON, New South Wales (AU); Meilang XUE, New South Wales (AU)

(21) Appl. No.: 17/255,190

(22) PCT Filed: Jun. 28, 2019
(86) PCT No.: PCT/AU2019/050691

§ 371 Date: Dec. 22, 2020

Related U.S. Application Data
(60) Provisional application No. 62/691,512, filed on Jun. 28, 2018.

(51) Int. Cl. A61K 38/17; A61K 38/08

(52) U.S. Cl. CPC A61K 38/177; A61K 38/08

(57) ABSTRACT

The invention relates to a method of minimising a symptom of dermatitis in an individual having a skin disease or condition comprising the administration of a TR47 related peptide, wherein the TR47 related peptide generally refers to a peptide having an N-terminal sequence NH₂-NPND or NH2-NPNDKY, which is derived from the N-terminal sequence of the Met1-Arg46 deleted human PAR1 (protease activated receptor 1) sequence.

### FIELD OF THE INVENTION

The invention relates to inflammatory skin disorders and to methods for treatment of same.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

PAR-1 has been considered as a potential target for development of inhibitors of inflammation. This is because in some inflammatory conditions, PAR-1 is activated resulting in the generation of pro-inflammatory signals.

The mechanism of action is believed to be that thrombin cleaves PAR-1 to generate an S⁴²FLLRN N-terminus which ostensibly acts as a tethered ligand for binding to loop 2 and likely other portions of PAR-1. The binding of the tethered ligand to loop 2 leads to PAR-1 conformation changes, rapid internalisation to the cytosol and concomitant generation of pro-inflammatory signals [Coughlin S R and Camerer E 2003 J. Clin. Investig 111:25-27; Ramachandran R. et al. 2012 Nature Reviews Drug Discovery 11: 69:86.

One outcome of the structural changes to PAR-1 arising from protease cleavage of PAR-1 (especially by thrombin) is that PAR-1 activation is irreversible [Trejo, J. 2003 J. Pharmacol and Exper Therap 307:437-442].

A range of compounds have been proposed to inhibit inflammation by antagonising the development of PAR-1 related pro-inflammatory signals. One approach has been to block signal development by blocking the extracellular domains for PAR-1, for example, using thrombostatins and modified bradykinin derived blocking peptides [Derian C K et al. 2003 Expert Opin Investig Drugs 12:209-221].

Another approach has been to develop monoclonal antibodies against the cleavage site of PAR 1 to block cleavage and activation of PAR-1 [O'Brien P J et al. 2001 Oncogene 20:1570-1581].

In another approach, small molecule PAR-1 antagonists have been generated based on the structure of the tethered ligand for PAR-1. These small molecule antagonists function by blocking interaction of the tethered ligand with binding sites on the extracellular face of the receptor but do not inhibit thrombin binding or receptor cleavage [Hollenberg M D and Compton S J 2002 Pharmacol Rev 54:203-217].

PAR-1 activation may also lead to the transmission of anti-inflammatory signals. It has been shown that this activation can involve cleavage of PAR-1 by APC to form a PAR-1 receptor having an N-terminus of N⁴⁷PNDKY [Mosnier L O et al. 2012 Blood 120:5237-5246]. The substrate for the reaction for the production of N-terminal N⁴⁷PNDKY is non-cleaved PAR-1. In particular, PAR-1 having the N-terminus of N⁴⁷PNDKY is not generated if PAR-1 has been prior cleaved by another enzyme such as thrombin [Mosnier L O et al. 2012 Blood 120:5237-5246].

In studies performed in vitro in artificial systems using endothelial cells, thrombin has a much higher kinetic efficiency for cleavage of PAR-1 than does APC [Ludeman M J. Et al. 2005 J. Biol. Chem 280:13122-8]. The extension of this is that where thrombin is in a higher relative abundance, PAR-1 is irreversibly activated for transmission of pro-inflammatory signals.

It has been reported that a peptide having a PAR-1 sequence with an N-terminus of N⁴⁷PNDKY may have anti-inflammatory effects in neural cells in in vitro conditions [Gorbacheva L R et al. 2017 Biochemistry (Moscow) 82:778-790]. As discussed, these peptides are unlikely to have anti-inflammatory effects on PAR-1 receptors that have been irreversibly activated by thrombin.

To summarise the above, PAR-1 is considered as a potential therapeutic target for treatment of some inflammatory disorders, either to minimise pro-inflammatory signals or to maximise anti-inflammatory signals, and a number of different approaches dependent of the structure/function relationships of the variously activated forms of PAR-1 are under consideration for exploring this potential.

Inflammatory disorders of the skin may present as rashes and related skin lesions with epidermal involvement. These include eczematous, scaling, blistering, papular, pustular and hypopigmented lesions.

Human tissue kallikreins (KLKs), especially KLK5 and KLK14 are skin serine proteases located in the subcutaneous layer and having a role in desquamation [Lee S E et al. 2014 Yonsei Med J 51: 808-822]. These enzymes are known to cleave protease receptors, including PAR-1, PAR-2 at the same site recognised by trypsin and/or thrombin [Oikonomopoulou K et al 2006 Biol Chem 387: 817-824] leading to production of inflammatory signals and indeed it is known that KLK5 and KLK14 activation of PAR-2 activation leads various symptoms of skin inflammation including itch [Murota and Katayama 2017 Allergology International 66: 8-13].

There is a need for new methods and compositions for minimisation of symptoms of skin inflammation, including inflammation associated with eczematous skin disorders such as allergic contact dermatitis.

### SUMMARY OF THE INVENTION

In one embodiment there is provided a method for minimising a symptom of dermatitis in an individual having a skin disorder including administering a therapeutically effective amount of a TR47 related peptide to the individual, thereby minimising a symptom of dermatitis in the individual.

In another embodiment there is provided a method for minimising skin itch, pain, swelling or redness in an individual having a skin disorder including administering a therapeutically effective amount of a TR47 related peptide to the individual, thereby minimising skin itch, pain, swelling or redness in the individual.

In another embodiment there is provided a method for minimising the size or appearance of a rash in an individual having a skin disorder including administering a therapeutically effective amount of a TR47 related peptide to the individual, thereby minimising the size or appearance of a rash in the individual.

In the above described embodiments, it is preferred that the skin disorder is a condition having, or arising from, epidermal involvement or injury.

In the above described embodiments it is preferred that the skin disorder is associated with eczematous rash or lesion.

In the above described embodiments, the skin disorder may be selected from the group consisting of atopic dermatitis, contact dermatitis, allergic dermatitis, acne, rosacea, psoriasis, chronic wounds, blistering diseases and toxic epidermal necrolysis, and the administration of the TR47 related peptide at least minimises the progression of the disorder.

In the above described embodiments, the skin disorder is preferably associated with expression or production of a serine protease, preferably kallikrein (KLK), preferably KLK5 or KLK14, preferably over production or over expression of KLK.

Preferably the region of skin disorder or lesion comprises KLK activity arising from over expression or over production of KLK at the time of administration of the TR47 related peptide.

In another embodiment there is provided a method for minimising a symptom of dermatitis in an individual having a skin disorder including administering to the individual a therapeutically effective amount of:
a TR47 related peptide and
a reagent for minimising inflammation, thereby minimising a symptom of dermatitis in the individual.

In the above described embodiments, the TR47 related peptide may be administered in a form enabling topical dermal application, such as a gel.

In one embodiment there is provided a method for minimising the production or expression of a molecule selected from the group consisting of TNFα, IL-1, IL-6, IL-17 and IL-23 by a cell, preferably an epidermal cell including the step of contacting an epidermal cell with a TR47-related peptide, thereby minimising the production or expression of TNFα, IL-1, IL-6, IL-17 and IL-23 by an epidermal cell.

In another embodiment there is provided a method for increasing the production or expression of MMP-2 by a cell, preferably an epidermal cell including the step of contacting an epidermal cell with a TR47-related peptide, thereby increasing the production or expression of MMP-2 by an epidermal cell.

In another embodiment there is provided a method for inducing phosphorylation of Akt Ser⁴⁷³ in a cell, preferably an epidermal cell including the step of contacting an epidermal cell with a TR47-related peptide, thereby inducing phosphorylation of Akt (Protein Kinase B) Ser⁴⁷³ by an epidermal cell.

A skin disorder, lesion or dermatitis may have, at the time of administration of the TR47 related peptide, an abnormal amount of kallikrein, especially KLK5 and/or KLK14 activity, and in particular, an amount of kallikrein activity not observed in skin not having dermatitis or related inflammation. Kallikrein activity may be determined by the skilled worker. See for example Lee S et al. supra; Murota and Katayama supra. One outcome of the kallikrein activity may be the cleavage of PAR-1 receptors on cells located in the region of inflammation by kallikrein, thus producing the pro-inflammatory S⁴²FLLRN N terminus and induction of inflammation in, or by these cells. The production of the S⁴²FLLRN N terminus can be determined by methods known to the skilled worker. See Coughlin and Camerer supra; Ramachandran et al. supra. A TR47 related peptide may be administered to the disorder, lesion or region of dermatitis in these circumstances and enable generation of anti-inflammatory signals. An anti-inflammatory signal may be identified by assessing for anti-inflammatory signalling through the Akt signalling pathway, on the basis of phosphorylation of Akt, for example at Ser⁴⁷³. An increase in Akt phosphorylation generally indicates the formation of an anti-inflammatory response. Akt phosphorylation may be assessed by methods known to the skilled worker, and as exemplified herein. Thus in another embodiment there is provided a method for treatment of an individual for a skin disorder or skin lesion, preferably an inflammatory skin disorder or lesion, the disorder or lesion having an overproduction or overexpression of a kallikrein, preferably kallikrein 5 or kallikrein 14, the overproduction or overexpression of kallikrein providing for, or enabling cleavage of PAR-1 receptors of cells in the disorder or lesion, the method including the step of: topically administering a TR47 related peptide, preferably a TR47 related peptide consisting of SEQ ID No: 17, preferably by administration of a gel, ointment, paste, or injection comprising the TR47 related peptide to the disorder or lesion, to provide for, or to enable phosphorylation of Akt, preferably phosphorylation of Akt Ser⁴⁷³ of a cell, or to provide for, or to enable anti-inflammatory signalling through the Akt signalling pathway in a cell, the cell located in the region of the skin disorder or skin lesion, thereby treating the individual for the skin disorder or lesion.

In another embodiment there is provided a method for minimising the proliferation of a cell, preferably an epidermal cell including the step of contacting an epidermal cell with a TR47-related peptide, thereby minimising the proliferation of an epidermal cell.

In the above described embodiments, the epidermal cell may be a keratinocyte or a cell of a neighbouring tissue including a dermal fibroblast, a dermal monocyte, or a dermal macrophage.

In the above described embodiments, the TR47 related peptide may be contacted with the cell, preferably an epidermal cell, in the presence of a serine protease, preferably a kallikrein, more preferably kallikrein 5 or kallikrein 14.

In a further embodiment there is provided an composition formulated for topical administration, preferably in the form of a gel, paste or ointment including a therapeutically effective amount of a TR47 related peptide and a pharmaceutically acceptable diluent, solvent or excipient for enabling topical administration of the composition to skin.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Amino acid sequence of PAR 1 (SEQ ID NO: 1)

FIG. 2: Amino acid sequence of 10 mer TR47 related peptide (SEQ ID NO: 2)

FIG. 3: Amino acid sequence of 20 mer TR47 related peptide (SEQ ID NO: 3)

FIG. 4: Amino acid sequence of 30 mer TR47 related peptide (SEQ ID NO: 4)

FIG. 5: Tetra-branched TR47 related peptide (SEQ ID NO: 5)

FIG. 6: Amino acid sequence of TRAP peptide (SEQ ID NO: 6)

FIG. 7: Amino acid sequence of Akt (SEQ ID NO: 7)

FIG. 8: A) Relative p-AKT activity in presence of PAR1 peptide and thrombin. B) Relative p-AKT activity in the presence PAR1 peptide following thrombin treatment, and in presence of PAR1 peptide prior to thrombin treatment.

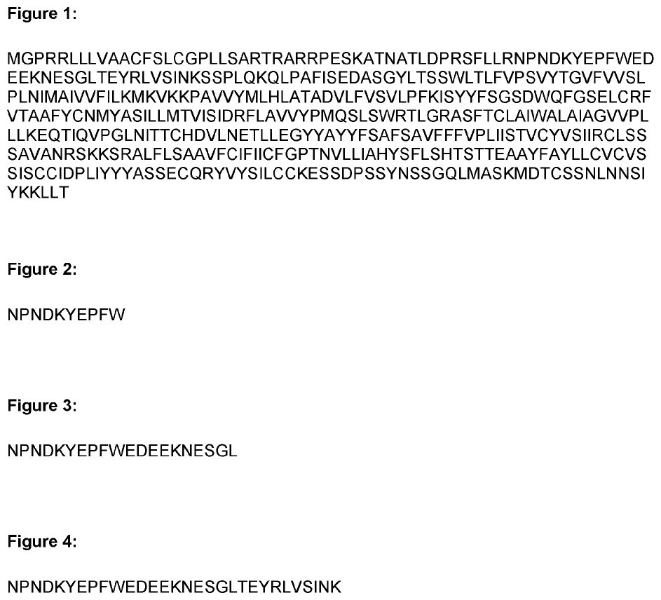

### DETAILED DESCRIPTION OF THE EMBODIMENTS

The invention generally relates to improvements in the management of skin diseases and conditions, the improvements arising from administration of a TR47 related peptide. It is believed that those improvements may be observed at the cellular level including minimisation of production or expression of inflammatory mediators, and at a clinical level in terms of minimisation of key symptoms of dermatitis.

The invention is applicable to a skin disease or condition involving dermatitis generally where there are common clinical manifestations of inflammation of skin tissue, especially epidermal tissue. Those symptoms or manifestations are described in more detail below and include skin redness, swelling, itch, pain and heat.

In preferred embodiments, the invention is applicable to skin disease or conditions where dermatitis arises as a condition that is secondary to another disease or condition. For example the skin disease or condition may arise from infection, cancer or autoimmunity. In other embodiments, the skin disease or condition arises as a primary disease of the skin, in which case the initial injury is to skin tissue and the disease may more or less localise to the skin.

A common feature of skin inflammation, particularly dermatitis presenting with epidermal involvement, is the role of the cells of the epidermis, especially the keratinocytes. These cells are understood to be a key source of inflammatory mediators such as TNF-α, IL-1 and IL-6. It is believed that the anti-inflammatory effects concomitant with the application of TR47 related peptides according to the invention arise from modification of the amount of expression or production of these anti-inflammatory mediators.

While not wanting to be bound by hypothesis, it is believed that an anti-inflammatory signal is provided in inflammatory keratinocytes by TR47 related peptide through contact of the TR47 related peptide with keratinocyte PAR-1 receptors. It is a surprising finding of the invention that such a signal can be generated by TR47 related peptides where it had been understood that keratinocyte PAR-1 receptors are prior cleaved by kallikreins and a range of other skin-expressed serine proteases, and therefore irreversibly activated for formation of a pro-inflammatory signal in epidermal cells. In contrast, it is believed that the invention has shown that epidermal cell PAR-1 receptors whether prior activated by kallikreins or not, can be activated for formation of an anti-inflammatory signal by TR47 related peptides.

“Dermatitis” is a commonly known inflammatory condition primarily affecting cells of the skin, especially the epidermal layer, especially causing injury to keratinocytes, squamous epithelial cells, fibroblasts and capillary endothelial cells.

“A symptom of dermatitis” generally refers to one or more of the following manifestations: skin and epidermal swelling, skin redness, skin itch, skin pain and local skin hyperthermia.

A “rash” generally refers to a skin lesion that present with some or all of the above mentioned symptoms of dermatitis. A rash may be an eczematous, scaling, blistering, papular, pustular or hypopigmented epidermal lesion. A rash may be visually observed on the basis of localised skin or epidermal swelling and or localised skin redness.

“A skin disease or condition” generally refers to a pathology of the skin organ itself. The pathology may be primary, i.e. having aetiology in the skin proper, or it may be secondary i.e. arising from an aetiology in another tissue or organ which injures the skin. A skin disease or condition may or may not be an inflammatory condition of the skin.

“An inflammatory skin disease or condition” generally refers to a pathology wherein inflammation is a key contributor to the development of the disease or condition. Generally, in such a condition, treatment of an anti-inflammatory agent, such as a steroid, has effect of minimising or reducing the pathology.

“Minimising a symptom” generally refers to at least reducing symptom severity, for example, reducing skin and epidermal swelling, skin redness, skin itch, skin pain and local skin hyperthermia. It does not necessarily mean ablating a symptom.

“Minimising the size or appearance of rash” generally refers to minimising the skin redness of the rash or minimising the diameter of the rash. Where the rash is associated with scaling, blistering or another morphological feature, it may involve the minimisation of that feature.

A “TR47-related peptide” generally refers to a peptide having an N terminal sequence NH₂-NPND or NH₂-NPNDKY.

“Akt” generally refers to a polypeptide having a sequence shown in SEQ ID No:

A “therapeutically effective amount” generally refers to an amount of TR47 related peptide that is effective for minimising one or more symptoms of an inflammatory skin disease, or for minimising the production or expression of inflammatory mediators from a keratinocyte or other epidermal cell.

“Comprise” and variations of the term, such as “comprising”, “comprises” and “comprised”, are not intended to exclude further additives, components, integers or steps unless the context requires otherwise.

In one embodiment there is provided a method for minimising a symptom of dermatitis in an individual having a skin disease or condition including administering a therapeutically effective amount of a TR47 related peptide to the individual, thereby minimising a symptom of dermatitis in the individual.

In one embodiment there is provided a method for minimising skin redness in an individual having a skin disease or condition including administering a therapeutically effective amount of a TR47 related peptide to the individual, thereby minimising skin redness in the individual.

In one embodiment there is provided a method for minimising skin swelling in an individual having a skin disease or condition including administering a therapeutically effective amount of a TR47 related peptide to the individual, thereby minimising skin swelling in the individual.

In one embodiment there is provided a method for minimising skin itch in an individual having a skin disease or condition including administering a therapeutically effective amount of a TR47 related peptide to the individual, thereby minimising skin itch in the individual.

In one embodiment there is provided a method for minimising localised skin hyperthermia in an individual having a skin disease or condition including administering a therapeutically effective amount of a TR47 related peptide to the individual, thereby minimising localised skin hyperthermia in the individual.

In one embodiment there is provided a method for minimising the size or appearance of a rash in an individual having a skin disease or condition including administering a therapeutically effective amount of a TR47 related peptide to the individual, thereby minimising the size or appearance of a rash in the individual.

In one embodiment there is provided a method for minimising skin blistering or scaling in an individual having a skin disease or condition including administering a therapeutically effective amount of a TR47 related peptide to the individual, thereby minimising skin blistering or scaling in the individual.

In one embodiment there is provided a method of minimising dermatitis in an individual, including administering a therapeutically effective amount of a TR47 related peptide to the individual, thereby minimising dermatitis in the individual. The dermatitis may be allergic contact dermatitis.

In a particularly preferred embodiment, the TR47 related peptide is given at an early stage of development of skin disease or condition, preferably prior to development of fibrotic skin tissue.

The TR47 related peptide may be administered to a skin disorder or skin lesion that contains an increased or abnormal amount of serine protease activity, preferably kallikrein activity, more preferably kallikrein 5 or kallikrein 14 activity.

In certain embodiments, a TR47 related peptide may be administered with another therapeutic compound indicated for treatment of a skin disease or condition, especially for treatment of inflammatory skin disease or condition such as allergic contact dermatitis. Examples include steroids and inflammatory cytokine inhibitors such as antibodies and other cytokine antagonists. Thus the invention further relates to a method for minimising a symptom of dermatitis in an individual having a skin condition or disease including administering to the individual a therapeutically effective amount of:
a TR47 related peptide and
an anti-TNF, or anti IL-13 or anti IL-27 antibody for minimising inflammation, thereby minimising a symptom of dermatitis in the individual.

As described further herein, the TR47 related peptide may be administered topically to the dermal surface in the form of a gel.

The invention further relates to utilising TR47 related peptides to minimise the production or expression of inflammatory mediators by epidermal cells, in particular by keratinocytes, squamous epithelial cells, and cells of neighbouring tissue including fibroblasts and capillary endothelial cells. These embodiments may also be useful in ex vivo applications for conditioning cells so that they define an anti-inflammatory profile, or in in vitro embodiments for monitoring or determining minimisation of inflammation in cells obtained from a diseased or inflamed skin.

In accordance with the above, in one embodiment there is provided a method for minimising the production or expression of a molecule selected from the group consisting of TNFα, IL-1 and IL-6, by a epidermal cell including the step of contacting an epidermal cell with a TR47-related peptide, thereby minimising the production or expression of TNFα, IL-1 and IL-6 by the epidermal cell.

In another embodiment there is provided a method for inducing phosphorylation of Akt Ser⁴⁷³ in an epidermal cell, particularly a keratinocyte, including the step of contacting an epidermal cell with a TR47-related peptide, thereby inducing phosphorylation of Akt Ser⁴⁷³ by an epidermal cell.

In another embodiment there is provided a method for minimising the proliferation of an epidermal cell including the step of contacting an epidermal cell with a TR47-related peptide, thereby minimising the proliferation of an epidermal cell.

In the above described embodiments, the epidermal cell may be a keratinocyte, a squamous epithelial cell, or a cell of a neighbouring tissue including a fibroblast and a capillary endothelial cell.

In another embodiment there is provided a TR47 related peptide or composition comprising same for use in minimising a symptom of dermatitis in an individual having a skin disease or condition, or for treating dermatitis, or for minimising the progression of dermatitis.

In another embodiment there is provided a TR47 related peptide, preferably a TR47 related peptide consisting of SEQ ID No:17 or composition comprising same for use in minimising or treating a skin disorder or skin lesion, the disorder or lesion having an overproduction or overexpression of a kallikrein, preferably kallikrein 5 or kallikrein 14, said overproduction or overexpression of kallikrein providing for or enabling cleavage of PAR-1 receptors, wherein the TR47 related peptide enables phosphorylation of Akt, preferably phosphorylation of Akt Ser⁴⁷³, by a cell in the region of the skin disorder or skin lesion.

In another embodiment there is provided use of a TR47 related peptide or composition comprising same for the manufacture of a medicament for use in minimising a symptom of dermatitis in an individual having a skin disease or condition, or for treating dermatitis, or for minimising the progression of dermatitis.

In another embodiment there is provided a use of a TR47 peptide or composition comprising same for minimising a symptom of dermatitis in an individual having a skin disease or condition, or for treating dermatitis, or for minimising the progression of dermatitis.

The invention provides TR47 related peptides which are generally anti-inflammatory to the extent that they antagonise the production, expression or action of inflammatory mediators. These peptides generally activate the PI3k-Akt signalling pathway, inhibit secretion of TNF-α by epidermal cells, and reduce epidermal cell NFκB activation.

Typically, a TR47 related peptide has an N terminal sequence of at least NPND, or an N terminal sequence that is homologous to NPND.

The TR47 peptide, including NPND, may contain a total of 4, 5, 6, 7, 8, 9, 10, 15, 20, 50, 100, 200, 300 or more amino acid residues in length. Some of the polypeptides comprise from about 4 amino acid residues to about 100 amino acid residues. Some of the polypeptides comprise from about 6 amino acid residues to about 50 amino acid residues.

In one embodiment, the TR47 related peptide has an amino acid sequence selected from the group consisting of: NPND (SEQ ID No: 11), NPNDK (SEQ ID No: 12), NPNDKY (SEQ ID No: 13), NPNDKYE (SEQ ID No: 14), NPNDKYEP (SEQ ID No: 15) and NPNDKYEPF (SEQ ID No: 16).

In one embodiment, the TR47 related peptide has an amino acid sequence selected from the group consisting of NPNDX-Y wherein:
X-Y is a sequence from K⁵¹ of SEQ ID NO:1 to L⁶⁶ of SEQ ID NO:1;
X-Y is a sequence from K⁵¹ of SEQ ID NO:1 to L⁹⁶ of SEQ ID NO:1;
X-Y is a sequence from K⁵¹ of SEQ ID NO:1 to T¹⁴⁶ of SEQ ID NO:1;
X-Y is a sequence from K⁵¹ of SEQ ID NO:1 to V²⁴⁵ of SEQ ID NO:1; and
X-Y is a sequence from K⁵¹ of SEQ ID NO:1 to T³⁴⁶ of SEQ ID NO:1.

A few specific examples of the PAR 1-derived cytoprotective polypeptides of the invention are shown in Table 1:

| SEQ ID No | Sequence |
|---|---|
| 2 | NPNDKYEPFW |
| 3 | NPNDKYEPFWEDEEKNESGL |
| 4 | NPNDKYEPFWEDEEKNESGLTEYRLVSINK |
| 8 | NPNDKY |
| 9 | NPNDKYEPFWEDE |
| 10 | NPNDKYEPFWEDEEKN |

In a preferred embodiment, the TR47 related peptide has the sequence of SEQ ID No: 17: NPNDKYEPFWED

In some embodiments, a TR47 related peptide has at least 70%, preferably 80%, preferably 90%, preferably 95%, preferably 96%, 97%, 98%, or 99% identity to a peptide shown in Table 1, provided that the peptide has the N terminal sequence NPND. Percent sequence identity may be determined by conventional methods, by means of computer programs known in the art such as GAP provided in the GCG program package (Program Manual for the Wisconsin Package, Version 8, August 1994, Genetics Computer Group, 575 Science Drive, Madison, Wis., USA 53711) as disclosed in Needleman, S. B. and Wunsch, CD., (1970), Journal of Molecular Biology, 48, 443-453, which is hereby incorporated by reference in its entirety. GAP is used with the following settings for polypeptide sequence comparison: GAP creation penalty of 3.0 and GAP extension penalty of 0.1.

In some embodiments, the TR47 related peptides can contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by polypeptide bonds. Similarly, they can also be cyclic polypeptides and other conformationally constrained structures. Methods for modifying a polypeptide to generate analogs and derivatives are well known in the art, e.g., Roberts and Vellaccio, The Peptides: Analysis, Synthesis, Biology, Eds. Gross and Meinhofer, Vol. 5, p. 341, Academic Press, Inc., New York, N.Y. (1983); and Burger's Medicinal Chemistry and Drug Discovery, Ed. Manfred E. Wolff, Ch. 15, pp. 619-620, John Wiley & Sons Inc., New York, N.Y. (1995).

Some other derivative compounds of the TR47 related peptides are peptidomimetics. Peptidomimetics based on TR47 related peptides substantially retain the activities of the TR47 related peptide. They include chemically modified polypeptides, polypeptide-like molecules containing non-naturally occurring amino acids, peptoids and the like, have a structure substantially the same as the reference polypeptides upon which the peptidomimetic is derived (see, for example, Burger's Medicinal Chemistry and Drug Discovery, 1995, supra). For example, the peptidomimetics can have one or more residues chemically derivatized by reaction of a functional side group. In addition to side group derivatizations, a chemical derivative can have one or more backbone modifications including alpha-amino substitutions such as N-methyl, N-ethyl, N-propyl and the like, and alpha-carbonyl substitutions such as thioester, thioamide, guanidino and the like. Typically, a peptidomimetic shows a considerable degree of structural identity when compared to the reference polypeptide and exhibits characteristics which are recognizable or known as being derived from related to the reference polypeptide, Peptidomimetics include, for example, organic structures which exhibit similar properties such as charge and charge spacing characteristics of the reference polypeptide. Peptidomimetics also can include constrained structures so as to maintain optimal spacing and charge interactions of the amino acid functional groups.

In some other embodiments, the TR47 related peptide can be dimerized or multimerized by covalent attachment to at least one linker moiety. For example, the peptides or polypeptides can be conjugated with a C₁₋₁₂ linking moiety optionally terminated with one or two —NH-0 linkages and optionally substituted at one or more available carbon atoms with a lower alkyl substituent. The TR47 related peptide described herein can be joined by other chemical bond linkages, such as linkages by disulfide bonds or by chemical bridges. In some other embodiments, the TR47 related peptide can be linked physically in tandem to form a polymer of TR47 related peptides.

The peptides making up such a polymer can be spaced apart from each other by a peptide linker. In some embodiments, molecular biology techniques well known in the art can be used to create a polymer of TR47 related peptides. In some embodiments, polyethylene glycol (PEG) may serve as a linker that dimerizes two peptide monomers. For example, a single PEG moiety containing two reactive functional groups may be simultaneously attached to the N-termini of both peptide chains of a peptide dimer. These peptides are referred to herein as “PEGylated peptides.” In some embodiments, the peptide monomers of the invention may be oligomerized using the biotinistreptavidin system.

In one embodiment, the TR47 related peptide comprises at least 2, preferably 3, preferably 4, preferably 5 or more TR47 related peptide sequences.

In one embodiment the TR47 related peptide is defined by the general formula:

A-L₁-B-L₂-C

wherein each of A, B and C are C₁-C₁₂ alkyl;

wherein each of A, B and C are substituted with one or more peptides having an amino acid sequence selected from the group consisting of SEQ ID No: 2 to 4, or 8 to 10; and

wherein L₁ and L₂ are each independently linker groups.

Preferably L₁ and/or L₂ comprise an amide group,

In a particularly preferred embodiment, the TR47 related peptide for use in a method described herein has a structure shown below:

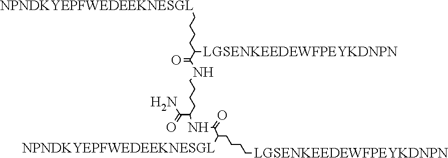

Methods for stabilizing peptides known in the art may be used with the methods and compositions described herein. For example, using D-amino acids, using reduced amide bonds for the peptide backbone, and using non-peptide bonds to link the side chains, including, but not limited to, pyrrolinone and sugar mimetics can each provide stabilization. The design and synthesis of sugar scaffold peptide mimetics are described in the art, e.g., Hirschmann et al., J. Med. Chem. 36, 2441-2448, 1996. Further, pyrrolinone-based peptide mimetics present the peptide pharmacophore on a stable background that has improved bioavailability characteristics (see, e.g., Smith et al. J. Am. Chem. Soc. 122, 11037-11038, 2000).

In some embodiment, derivative compounds of the TR47 related peptides include modifications within the sequence, such as, modification by term inal-NH₂ acylation, e.g., acetylation, or thioglycolic acid amidation, by terminal-carboxylarnidation, e.g., with ammonia, methylamine, and the like terminal modifications. One can also modify the amino and/or carboxy termini of the polypeptides described herein. Terminal modifications are useful to reduce susceptibility by proteinase digestion, and therefore can serve to prolong half-life of the polypeptides in solution, particularly in biological fluids where proteases may be present. Amino terminus modifications include methylation (e.g., —NHCH₃ or —N(CH₃)₂), acetylation (e.g.; with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In some embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the peptides described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art, for example, in U.S. Patent Application No. 20090035814; and Muralidharan and Muir, Nat. Methods, 3:429-38, 2006. C-terminal functional groups of the peptides described herein include amide, amide lower alkyl, amide di(lower alkyl), lower alkoxy, hydroxy, and carboxy, and the lower ester derivatives thereof, and the pharmaceutically acceptable salts thereof.

The TR47 related polypeptides described herein, including variants and derivatives thereof, can be chemically synthesized and purified by standard chemical or biochemical methods that are well known in the art. Some of the methods for generating analog or derivative compounds of the TR47 related polypeptides are described above. Other methods that may be employed for producing the TR47 related polypeptides and their derivative compounds, e.g.; solid phase peptide synthesis, are discussed below. For example, the peptides can be synthesized using t-Boc (tert-butyloxycarbonyl) or FMOC (9-flourenylmethloxycarbonyl) protection group described in the art. See, e.g., “Peptide synthesis and applications” in Methods in molecular biology Vol. 298, Ed. by John Howl; “Chemistry of Peptide Synthesis” by N. Leo Benoiton, 2005, CRC Press, (ISBN-13: 978-1574444544); and “Chemical Approaches to the Synthesis of Peptides and Proteins” by P. Lloyd-Williams, et. al., 1997, CRC-Press, (ISBN-13: 978-0849391422), Methods in Enzymology, Volume 289: Solid-Phase Peptide Synthesis, J. N. Abelson, M. I. Simon, G. B. Fields (Editors), Academic Press: 1st edition (1997) (ISBN-13: 978-0121821906); U.S. Pat. Nos. 4,965,343; and 5,849,954.

Solid phase peptide synthesis; developed by R. B. Merrifield, 1963, J. Am. Chem. Soc. 85 (14): 2149-2154, was a major breakthrough allowing for the chemical synthesis of peptides and small proteins. An insoluble polymer support (resin) is used to anchor the peptide chain as each additional alpha-amino acid is attached. This polymer support is constructed of 20-50 μm diameter particles which are chemically inert to the reagents and solvents used in solid phase peptide synthesis. These particles swell extensively in solvents, which makes the linker arms more accessible. Organic linkers attached to the polymer support activate the resin sites and strengthen the bond between the alpha-amino acid and the polymer support. Chloromethyl linkers, which were developed first, have been found to be unsatisfactory for longer peptides due to a decrease in step yields. The PAM (phenylacetamidomethyl) resin, because of the electron withdrawing power of the acid amide group on the phenylene ring, provides a much more stable bond than the classical resin. Another alternative resin for peptides under typical peptide synthesis conditions is the Wang resin. This resin is generally used with the FMOC labile protecting group.

A labile group protects the alpha-amino group of the amino acid. This group is easily removed after each coupling reaction so that the next alpha-amino protected amino acid may be added. Typical labile protecting groups include t-Boc (tert-butyloxycarbonyl) and FMOC. t-Boc is a very satisfactory labile group which is stable at room temperature and easily removed with dilute solutions of trifluoroacetic acid (TEA) and dichloromethane. FMOC is a base labile protecting group which is easily removed by concentrated solutions of amines (usually 20-55% piperidine in N-methylpyrrolidone). When using FMOC alpha-amino acids, an acid labile (or base stable) resin, such as an ether resin, is desired.

The stable blocking group protects the reactive functional group of an amino acid and prevents formation of complicated secondary chains. This blocking group must remain attached throughout the synthesis and may be removed after completion of synthesis. When choosing a stable blocking group, the labile protecting group and the cleavage procedure to be used should be considered. After generation of the resin bound synthetic peptide, the stable blocking groups are removed and the peptide is cleaved from the resin to produce a “free” peptide. In general, the stable blocking groups and organic linkers are labile to strong acids such as TFA. After the peptide is cleaved from the resin, the resin is washed away and the peptide is extracted with ether to remove unwanted materials such as the scavengers used in the cleavage reaction. The peptide is then frozen and lyophilized to produce the solid peptide. This is generally then characterized by HPLC and MALDI before being used. In addition, the peptide should be purified by HPLC to higher purity before use.

Commercial peptide synthesizing machines are available for solid phase peptide synthesis. For example, the Advanced Chemtech Model 396 Multiple Peptide Synthesizer and an Applied Biosystems Model 432A Peptide synthesizer are suitable. There are commercial companies that make custom synthetic peptides to order, e.g., Abbiotec, Abgent, AnaSpec Global Peptide Services, LLC., Invitrogen, and rPeptide, LLC.

The TR47 related polypeptides and derivatives thereof can also be synthesized and purified by molecular methods that are well known in the art. Recombinant polypeptides may be expressed in bacteria, mammal, insect, yeast, or plant cells.

Cell-free expression systems can also be used for producing TR47 related polypeptides of the invention. Cell-free expression systems offer several advantages over traditional cell-based expression methods, including the easy modification of reaction conditions to favor protein folding, decreased sensitivity to product toxicity and suitability for high-throughput strategies such as rapid expression screening or large amount protein production because of reduced reaction volumes and process time. The cell-free expression system can use plasmid or linear DNA. Moreover, improvements in translation efficiency have resulted in yields that exceed a milligram of protein per milliliter of reaction mix. An example of a cell-free translation system capable of producing proteins in high yield is described by Spirin et. al., Science 242:1162, 1988, The method uses a continuous flow design of the feeding buffer which contains amino acids, adenosine triphosphate (ATP), and guanosine triphosphate (GTP) throughout the reaction mixture and a continuous removal of the translated polypeptide product. The system uses E. coli lysate to provide the cell-free continuous feeding buffer. This continuous flow system is compatible with both prokaryotic and eukaryotic expression vectors. An example of large scale cell-free protein production is described in Chang et. al., Science 310:1950-3, 2005.

Other commercially available cell-free expression systems include the Expressway™ Cell-Free Expression Systems (Invitrogen) which utilize an E. coli-based in-vitro system for efficient, coupled transcription and translation reactions to produce up to milligram quantities of active recombinant protein in a tube reaction format; the Rapid Translation System (RTS) (Roche Applied Science) which also uses an E. coli-based in-vitro system; and the TNT Coupled Reticulocyte Lysate Systems (Promega) which uses a rabbit reticulocyte-based in-vitro system.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions.

A pharmaceutical composition of the invention may be formulated to enable administration by any known route. By way of example, the composition may be administered to enable administration by a mucosal, pulmonary, optical or other localized or systemic route (e.g., enteral and parenteral).

An injectable formulation of a TR47 related peptide may be supplied as a sterile, lyophilized powder for subcutaneous or epidermal injection including TR47 related peptide, sucrose, NaCl and sodium citrate. The vials may be reconstituted with sterile water for injection, USP, to give a concentration of about 100 mg/ml TR47 related peptide and this diluted TR47 related peptide may then be added to 0.9% Sodium Chloride Injection to give a concentration of from about 10 to about 50000 μg/ml TR-47 for administration to a patient. This is a particular preferred formulation for administration of TR47 by dermal injection.

Preferably the TR47 related peptide is provided in the form of an aqueous gel. An exemplary gel base may methylcellulose, alginate, carrageenan, dextran or glucan.

In certain embodiments, a pharmaceutical composition may include, in addition to a TR47 related peptide, one or more other agents for treatment of a skin disease or condition, especially an anti-inflammatory agent such as a steroid, or anti-inflammatory cytokine or anti-inflammatory antibody. In some embodiments, the one or more other agents for treatment of a skin disease or condition may be provided separately to the composition including the TR47 related peptide.

In one embodiment, the therapeutically effective amount of TR47 related peptide may be from 1 to 10,000 μg, preferably from 10 to 1000 μg per 10 cm² of the region of skin. A higher amount is generally preferred where the skin is more severely affected, or where the individual is at particular risk because of presence of local or systemic factors. Lower amounts may be preferred where the skin is not severely affected.

A therapeutically effective amount of TR47-related peptide for bolus administration, especially for intra-dermal injection can typically be 2 mg/kg or less, 1 mg/kg or less, 0.5 mg/kg or less, 0.04 mg/kg or less, 0.03 mg/kg or less, 0.02 mg/kg or less, 0.01 mg/kg or less, or 0.005 mg/kg or less. Typically, the therapeutic amount may be based on titering to a tissue level amount of about 0.01 μg/ml to about 1.6 μg/ml, preferably from about 0.01 μg/ml to about 0.5 μg/ml. It is also within the skill of the art to start doses at levels lower than required to achieve the desired therapeutic effect and to gradually increase the dosage until the desired effect is achieved. It is likewise within the skill of the art to determine optimal concentrations of variants to achieve the desired effects in the in vitro and ex vivo preparations of the invention. Depending on initial assay results, optimal concentrations can be in the range of, e.g., about 1-1,000 nM or about 1-200 μM depending on the general nature of the compound. In one embodiment, a therapeutically effective amount of TR47 related peptide may be 0.1-100 mg/kg.

The concentration of TR47 related peptide in the formulation may be between about 100 μg/ml and 5 mg/ml. In this embodiment, the volume of composition applied to the skin region may be about 100 μl to 5 ml.

The present disclosure includes methods of administering in vivo or ex vivo a TR47-related peptide described above (or compositions comprising a pharmaceutically acceptable excipient and one or more such peptides) to a subject, including, e.g., a mammal, including a human.

According to the invention, in in vivo methods, one or more cells or a population of cells of interest of the subject are contacted directly or indirectly with an amount of a TR47 related peptide effective in prophylactically or therapeutically treating the disease, disorder, or other condition. In direct contact/administration formats, the TR47 related peptide is typically administered or transferred directly to the cells to be treated or to the tissue site of interest by any of a variety of formats, including topical administration.

Topical treatment methods, for example, using a paste, gel, cream, oil, lotion, foam, ointment or like substance are particularly useful where the relevant skin region is one that contains a ruptured skin surface, as this permits penetration of the TR47 related peptide may be administered to the relevant strata of the skin tissue where the fibroblasts reside.

The composition may be provided to the skin generally with a sterile surface, such as a finger or spatula in a layer of no more than about 10 mm thickness, preferably about 3 mm thickness. It may then be rubbed or massaged into the skin region and surrounding area. The application is generally from once per day to once per week, and generally no longer than 20 weeks, or no longer than 12 weeks.

In one embodiment, the TR47 containing composition may be applied to a solid substrate i.e. a bandage, dressing or the like, and the substrate then fixed to the relevant skin region.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Peptides

Peptides used in this study are synthesised according to standard techniques to >95% purity.

### Example 2—In Vitro Keratinocyte Culture

Human keratinocytes are isolated from neonatal foreskin or discarded adult skin tissue after cosmetic surgery and cultured as described previously (Xue et al, Exp Cell Res 2004 299(1):119-27). Neonatal keratinocytes are cultured in keratinocyte serum free medium (K-SFM) supplemented with 5 ng/mL recombinant human EGF, 50 μg/mL bovine pituitary extract, 100 IU/mL penicillin and 100 IU/mL streptomycin. Adult keratinocytes are cultured in EpiLife™ Medium containing 60 μM calcium, 1 xHKGS, 100 IU/mL penicillin and 100 IU/mL streptomycin. Culture media are changed twice a week. When greater than 70% confluent, cultured cells are trypsinized and seeded into either 24 or 96-well culture plates, 24-well inserts or 8-well Permanox™ slides (Nalge Nunc International Corp) for test compounds treatment.

Conditionally immortalized human keratinocytes (HaCaT) are cultured and maintained in DMEM medium supplemented with 10% FBS and treated with test compounds and analysed similarly as primary cultured human keratinocytes.

### Example 3—Ex Vivo Keratinocyte Culture

Organotypic cultures are grown as described previously (Gangatirkar et al. Nat Protoc. 2007; 2:178-86.). Initially, rat tail collagen I matrices are neutralized using sodium hydroxide, and then mixed with FBS containing human foreskin fibroblasts (final concentration: 1×10⁵ cells/ml, 0.5 mg/ml collagen). The collagen-cells mixture is aliquoted to each insert (2 ml for each insert in a 6-well plate), polymerized at 37° C. for 2 hrs, and then allowed to contract for 2 days in DMEM with 10% FBS. After 2 days, 5×10⁵ keratinocytes are seeded on dermal equivalents, cultured submerged for 5 to 7 days, then raised to the air-liquid interface for another 5-7 days. During this period, organotypic cultures are treated with test compounds. After treatment, organotypic cultures are harvested for histological examination with hematoxylin and eosin staining or other functional/chemical analysis.

TR47 related peptides will enhance differentiation of cells to mimic that of skin. A well-structured epidermis will sit above and adhered to the dermis containing fibroblasts and collagen. Keratinocytes will form their typical multiple epidermal layers with a stratum basale on the lower layer and a cornified layer on top.

### Example 4—Proliferation Assay

Cell proliferation is detected by BrdU Cell Proliferation assay kit (Merck Millipore) according to manufacturer's instruction. Briefly, keratinocytes are seeded into a 96 well plate at 1×10⁵ cell/mL in 100 μL/well of culture medium. After overnight attachment, cells are treated with test compounds for 72 hrs. 6 hrs prior to the end of treatment, BrdU is incorporated into newly synthesized DNA strands of actively proliferating cells. After treatment, cells are fixed and DNA denatured. The BrdU is detected using anti-BrdU monoclonal antibody and final data detected using a spectrophotometer microplate reader set at dual wavelength of 450/550 nm.

TR47 related peptides are expected to dose-dependently increase proliferation of keratinocytes.

### Example 5—Cell Morphology and Death Assay

Cultured keratinocytes in 24 well plates are treated with test compounds. After the treatment, changes in cell morphology is examined under an inverted microscope. Cell apoptosis is examined using the annexin V-FITC apoptosis-detection kit according to the manufacturer's instructions. Briefly, cells are trypsinized, washed with flow cytometry staining buffer (FACS buffer, 5% FBS in phosphate-buffered saline (PBS), filtered), and stained with Fixable Viability dye. After staining, cells are washed twice with FACS buffer, stained with FITC conjugated Annexin V and finally analyzed with flow cytometry.

TR47 related peptides are expected to dose-dependently inhibit apoptosis of both cell types.

### Example 6—Quantitative Reverse Transcriptase-Polymerase Chain Reaction (qRT-PCR)

Total RNA is isolated from RSFs of passage 1 and passage 4 by using RNAzol (Molecular Research Centre, Cincinnati, Ohio, USA) according to the manufacturer's instructions (http://www.mrcgene.com/rnazol.htm). RNA concentration is determined by NanoDrop spectrophotometry (Thermo Scientific; Scoresby, Australia) and reverse-transcribed into complementary DNA (cDNA) using the cDNA synthesis kit (Bioline; Taunton, Mass., USA). Subsequently, qRT-PCR is performed with the Rotor-Gene 6000 Real-Time PCR machine (Corbett Life Science, Mortlake, Australia) by using ImmoMix (Bioline; Taunton) and SYBR Green dye (Qiagen, Hilden, Germany). The reaction mixture consists of 1 μg cDNA template, 0.75 μL each of forward and reverse primer, 12.5 μL ImmoMix and 2.5 μL SYBR Green. Cycling conditions comprise an initial activation step at 95° C. for 10 min followed by 45 amplification cycles of 95° C. for 15 s (denaturation), 58° C. for 20 s (primer annealing) and 20° C. for 45 s (extension). Specificity of the amplification reactions is verified by melting curve analysis.

Primers used in the assay are designed and checked for specificity by using the National Center for Biotechnology Information BLAST search tool (http://www.ncbi.nlm.nih.gov/tools/primer-blast). Data are analysed using the standard curve for absolute quantification method.

### Example 7—Histology

Skin tissue is fixed with 10% PBS buffered formalin (pH 7.4), embedded in paraffin, cut into 4 μm sections and mounted on slides. The sections are then deparaffinised via three changes of xylene for 2 min per change, through three changes of 100% ethanol for 2 min per change, one change each for 2 min for 95% ethanol, 70% ethanol, and finally rinsed in running tap water at room temperature for at least 2 min. Slides then were stained in hematoxylin solution for 3 min, washed under running tap water at room temperature for at least 5 min, and then stained in working eosin Y solution for 2 min. Slides were dehydrated, mounted and viewed using ausing light microscopy (BX-51/DP-72; Olympus, Tokyo, Japan). Resolution of dermatitis symptoms was determined using ImageJ software (National Institutes of Health, Bethesda, Md., USA).

### Example 8—Western Blotting

Briefly, cells are washed three times with PBS and lysed with lysis buffer (0.15 M NaCl, 0.01 mM PMSF, 1% NP-40, 0.02 M Tris, 6 M urea/H2O). Cell lysates are centrifuged at 10,000 g for 15 minutes and supernatants are separated by 10% sodium-dodecyl-sulphate polyacrylamide-gel electrophoresis (SDS-PAGE) and transferred to a PDVF membrane. The primary antibodies used are as follows against pan or phospho specific forms of Akt, GSK3β, ERK1/2. Immunoreactivity is detected by using the ECL detection system (Amersham Biosciences, Buckinghamshire, UK). Anti-human (3-actin (Sigma-Aldrich) antibody is included to normalize for unequal loading. Protein band intensity is evaluated by densitometry by ImageQuant™ LAS 4000 (GE Healthcare Life Sciences).

TR 47 related peptides are expected to dose-dependently increase phosphorylation of all three proteins depending on the level of confluence of either cell type.

### Example 9—ELISA

Levels of IL-6, IL-1 and TNFα are measured using ELISA kits in accordance with manufacturer's instructions. Briefly, samples are added to the ELISA plate that was previously coated with capture antibody, and after 1 to 2 hrs incubation, the samples are aspirated and the plate washed with wash buffer. Then a biotinylated detection antibody, linked directly to the primary antibody is added into each well in the plate and incubated for another 2 hrs. Finally the readout was obtained after incubation using using a spectrophotor, meter after incubation with horseradish peroxidase (HRP)-conjugate and a chromogenic substrate TMB (3, 3′, 5, 5′-tetramethylbenzidine) for HRP.

We expect that peptides will dose-dependently reduce the levels of each cytokine in both cell types.

### Example 10—Rac 1 Activation

Briefly, keratinocytes are grown in a 6 well plate till confluent and incubated in serum free medium without supplements overnight before addition of peptides (50 μM) for 30 or 180 minutes. Lysates (2 mg) are mixed with GST-PAK1 glutathione-agarose (150 μg) and after washing active GTP-Rac1 is eluted from GST-PAK1 glutathione-agarose by boiling in reducing SDS sample buffer. Active GTP-Rac1 is resolved on 12% SDSPAGE, transferred to PVDF membrane, and immunoblotted with a mouse anti-Rac1 antibody and anti-mouse secondary antibodies. Immunoblots are scanned and integrated fluorescence intensity units are quantified.

### Example 11—Statistical Analysis

The data are expressed as the mean±SD. Statistical analyses were performed by using the Student t test or analysis of variance (ANOVA) followed by the Bonferroni post hoc test (where appropriate). Statistical significance was accepted at the p<0.05 level.

### Example 12—Induction of Akt Activation in Keratinocytes

The TR47 peptide is to induce robust and sustained activation of Akt in keratinocytes as determined by phosphorylation of Ser473. To confirm activation of Akt by TR47, Akt-mediated inactivation of GSK3β via phosphorylation at Ser9 is determined. GSK3β is a well-known downstream substrate for Akt. TR47 is to induce significant Ser9-GSK3β phosphorylation with a time course that falls within the time course of TR47-mediated Akt activation. A scrambled control peptide is used to demonstrate no phosphorylation of Akt at Ser 473. A PAR-1 inhibitor SCH79797 is used to show that activation of Akt is dependent on PAR-1.

### Example 13—DNFB Model

6-8 weeks old female C57BL6 mice will be used. Contact hypersensitivity (CHS), a model of dermatitis, is induced by 2,4-dinitrofluorobenzene (DNFB) as described previously (Rose et al Exp Dermatol 2012, 21, 25-31). Mice are sensitized with DNFB on the shaved flank skin on day 0 and day 1, on the dorsum of both ears on day 5-13. Control mice receive same volume of solvent (acetone and olive oil (4:1, v/v) at the same time points. Severity of CHS is assessed by ear swelling measurement with a digital micrometer daily from day 5 to 14. Additionally tissue is fixed at days 5 to 14 for histology. For preventative treatment, the test compounds are delivered daily between day 2 and day 5 for four days via intraperitoneal (IP) injection/topical administration. For therapeutic treatment, the test compounds are delivered daily between day 8 and day 12, for five days. The test compounds are administered 2 hrs prior to DNFB treatment.

### Example 14—Assay for Swelling

This assay is performed as described by Thorne et al (Fundam Appl Toxicol 1991, 17, 790-806). 6 to 8 weeks old BALB/c mice will be used. At day 0: mouse abdomen is depilated and washed, then allowed to dry, then the induction dose of test compound is applied to abdomen in 100 ul volume. At day 5, baseline ear thickness is measured with a spring-loaded, low tension micrometer, three times for each ear. 40 ul of test compound diluted to highest non-irritating concentration is applied to the right ear, 40 ul of the vehicle to the left ear. Ear thickness is measured at 1, 24, 48, 72, and 96 hr post application and the ear thickness increase is the mean of 24, 48, and 72 hr values.

### Example 15—PAR-1 Peptide Treatment in Presence of and Prior to and Following Thrombin Treatment

Cells and Treatment:

AE.Hy926 cells (ATCC® CRL-2922′), the human umbilical vein cell line, established by fusing primary human umbilical vein cells with a thioguanine-resistant clone of A549 by exposure to polyethylene glycol (PEG), were cultured in Dulbecco's Modified Eagle's Medium (DMEM, high glucose) containing 10% fetal bovine serum (FBS) in 75 sq cm flasks. When cells reached confluence, they were trypsinzed and seeded into 48 well plates at 1×10⁴ viable cells/well for 2 days to reach complete confluency. Before treatment, cells were preincubated with DMEM medium (no FBS) for 2 hrs, then switched to fresh DMEM and treated with:
PAR1 12mer peptide (shown on graphs as PAR1) (or scrambled control (SC) of PAR1 peptide)
thrombin

either alone or at different combinations for 1 hr. After treatment, media were discarded and 100 μl lysis buffer was added to each wells and cell lysates were collected and stored in −40° C. for ELISA.

ELISA:

Human p-GSK and p-AKT were measured using ELISA Dueset kits (R&D Systems) according to manufacturers' instructions.

Results:

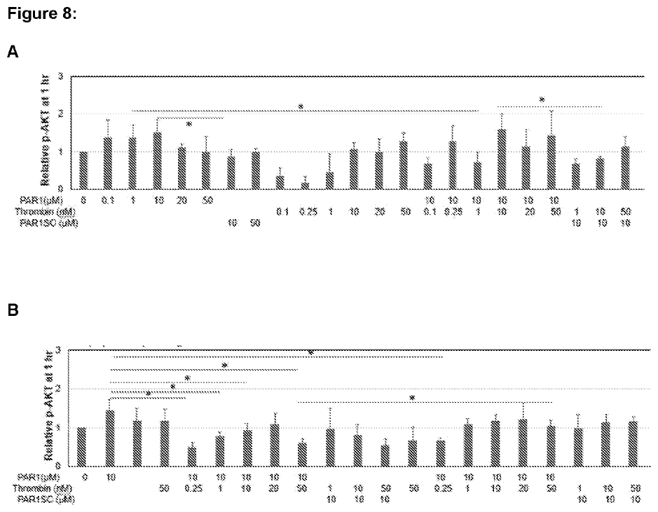

The PAR1 peptide induced robust activation of Akt as determined by phosphorylation of Ser⁴⁷³, after 1 hour. The AKT signaling pathway is synonymous with cytoprotection in endothelial cells [Akt mediates cytoprotection of endothelial cells by vascular endothelial growth factor in an anchorage dependent manner. Fujio Y, Walsh K. J Biol Chem. 1999 Jun. 4; 274(23):16349-54; Insulin stabilizes microvascular endothelial barrier function via phosphatidylinositol 3-kinase/Akt-mediated Rac1 activation. Gündüz D, Thom J, Hussain I, Lopez D, Härtel FV, Erdogan A, Grebe M, Sedding D, Piper H M, Tillmanns H, Noll T, Aslam M. Arterioscler Thromb Vasc Biol. 2010 June; 30(6):1237-45] and other cells including epidermal cells [Phosphoinositide kinase signaling to Akt promotes keratinocyte differentiation versus death Calautti E, Li J, Saoncella S, Brissette J L, Goetinck P F. J Biol Chem. 2005 Sep. 23; 280(38):32856-65] as it mediates barrier stabilization, and enhances an anti-inflammatory as opposed to an inflammatory phenotype. In contrast, the latter is induced by thrombin. FIG. 8A shows that the addition of thrombin alone at 0.1 and 0.25 nM inhibited phosphorylation of AKT. When lower doses (0.1. or 0.25 uM) of thrombin and PAR1 peptide were added together simultaneously there was increased pAKT compared to adding thrombin alone, indicating that the PAR1 peptide exerts cytoprotective AKT activity even in the presence of the potent inflammatory enzyme, thrombin. At some higher concentrations, eg 10 nM, thrombin in the presence of peptide also significantly enhanced pAKT. FIG. 8B shows the effect of adding the PAR1 peptide 15 mins before or after thrombin, and its ability to enhance the Akt activity.

1. A method for minimising a symptom of dermatitis in an individual having a skin disease or condition including administering a therapeutically effective amount of a TR47 related peptide to the individual, thereby minimising a symptom of dermatitis in the individual.

2. The method of claim 1 wherein the skin disease or condition presents with epidermal involvement.

3. The method of claim 1 wherein the skin disease or condition is a primary disease of the skin.

4. The method of claim 1 wherein the skin disease or condition is allergic contact dermatitis.

5. The method of claim 1 wherein the symptom of dermatitis is selected from the group consisting of localized heat, skin redness, itch, pain or swelling.

6. The method of claim 1 wherein the administration of the TR47 related peptide minimises the production or expression of a molecule selected from the group consisting of TNFα, IL-1 and IL-6 in the individual.

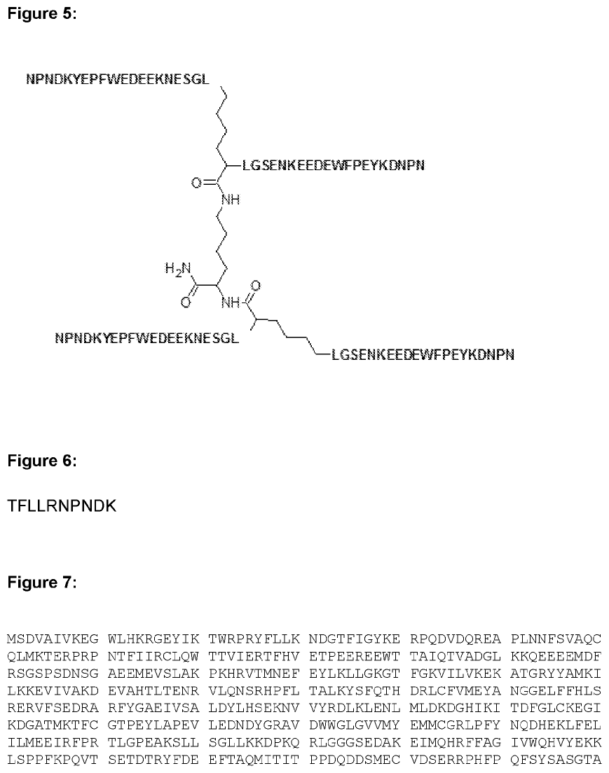

7. The method of claim 1 wherein the TR47 related peptide is selected from the group consisting of SEQ ID No: 2 to 4 or 8 to 17 or FIG. 5.

8. The method of claim 1 wherein the TR47 related peptide is administered in the form of a pharmaceutical composition.

9. The method of including the step of administering an anti-inflammatory compound to the individual.

10. The method of claim 1 wherein the TR47 related peptide is administered by topical application to the dermis.

11. The method of claim 1 wherein the skin disorder is dermatitis and the region of the skin of the individual having dermatitis has, at the time of the administration of the TR47 related peptide, an overexpression or overproduction of a kallikrein, said overexpression or overproduction of a kallikrein providing for, or resulting in, the cleavage of PAR-1 receptors on cells contained in the region of skin.

12. The method of claim 1 wherein the administration of a TR47 related peptide to a region of skin of the individual having the disorder enables, or causes, the phosphorylation of Akt, preferably phosphorylation of Akt Ser⁴⁷³, by a cell in the region of skin.
